(19) United States
(12) Patent Application Publication
(10) Pub. No.: US 2005/0261393 A1
(43) Pub. Date: Nov. 24, 2005

(54) Utilization of polyacids having a tight molar mass distribution

(76) Inventors: Markus Mikulla, Andechs-Frieding (DE); Gabriele Rackelmann, Gilching (DE); Klaus-Peter Stefan, Seefeld (DE)

Correspondence Address: CROWELL & MORING LLP;INTELLECTUAL PROPERTY GROUP, P.O. BOX 14300, WASHINGTON, DC, 20044-4300, US

(21) Appl. No.: 10/432,820

(22) PCT Filed: Nov. 19, 2001
(86) PCT No.: PCT/EP01/13350

§ 371 Date: Nov. 21, 2003

(30) Foreign Application Priority Data
Nov. 27, 2000 (DE) 100 58 829.8

(51) Int. Cl. A61F 2/00; C08K 3/00

(52) U.S. Cl. 523/113

(57) ABSTRACT

The invention relates to dental materials containing at least one polyacid or at least one polyacid and at least one salt of at least one polyacid with a molar mass distribution Mw/Mn ranging from 1.0 to 1.7, whereby the polyacid has a molecular weight Mw ranging from 1,000 to 500,000.

The invention relates to the use in dental materials of polyacids or of polyacids together with salts of polyacids with a narrow molar mass distribution.

Polyacids have been used in dental materials since about the end of the 1960s. For example, polyacrylic acid was used as ground substance in zinc polycarboxylate cements (D. C. Smith, Br. Dent. J., 125 (1968), 381-384) or also in glass ionomer cements (ASPA I, Wilson and Kent, 1969; DE 20 61 513 A).

While zinc polycarboxylate cements to this day are based on polyacrylic acid, the glass ionomer cements were developed further with regard to the chemical composition of the polyacid.

For example, copolymer acids of acrylic acid and itaconic acid for use in glass ionomer cements are known from S. Crisp, B. E. Kent, B. G. Lewis, A. J. Ferner and A. D. Wilson, J. Dent. Res., 59 (1980), 1055-1063. In addition, glass ionomer cements based on a copolymer of acrylic acid and maleic acid are known from EP 0 024 056 A1.

The use of polyvinylphosphonic acid is known, for example, from EP 0 340 016 A, EP 0 431 440 A, U.S. Pat. No. 5,179,135, GB 2 272 222 A and U.S. Pat. No. 5,601,640.

The use of copolymer acids of acrylic acid and vinyl-phosphonic acid is disclosed in GB 2 291 060 A and the use of polycarboxylic acids functionalized with amino acids is disclosed in U.S. Pat. No. 5,369,142.

The polyacids are usually used in dental materials, for example in polyelectrolyte cements and especially in zinc polycarboxylate cements and glass ionomer cements, as highly concentrated, for example approximately 30 to 50%, aqueous solutions.

The overall proportion of polyacid in dental cements is frequently, on the one hand, chosen to be as high as possible, in order to achieve maximum strength of the cured cement; on the other hand, the system has to exhibit, on mixing and applying, the lowest possible viscosity, guaranteeing that it can be removed from its container, for example from capsules. A reduction in the overall content of polyacid in dental cements is generally, and especially in reinforced glass ionomer cements, accompanied by an obvious deterioration in the mechanical strength of the cured cement.

That is why there exists a high demand for dental cements which have a reduced viscosity at a high content of polyacid.

Various starting points for the solution of this problem have in principle been known to date but they suffer from disadvantages.

The possibilities of inserting the polyacid in glass ionomer cements in powder form are limited, in particular in reinforced glass ionomer filling cements, since in the mixing operation only a very short time, for example up to a few minutes, is available for dissolution of the polyacid in the total system.

In principle, the viscosity of a polymer in solution can be reduced by using branched polymers. At the same molecular weight, the solution viscosity of polymers generally becomes smaller as the degree of branching increases. That is why liquid cements of low viscosity can be prepared by use of short-chain branched, long-chain branched, comb-like branched, star-shaped, hyperbranched or dendrimeric polyacids, which liquid cements favorably influence the overall viscosity of the system.

The disadvantage of such polyacids is, first, the significantly higher expenditure on synthesis and consequently the increased manufacturing costs, especially in the manufacture of specific branching structures, and, secondly, with increasing branching, the acid groups of the polyacid become more difficult to access, so that an undesired change in the setting characteristic occurs. In addition, poor accessibility of the acid groups results in lower degrees of crosslinking and consequently reduced strengths in the cement.

Investigations into the influence of the molar mass distribution on the properties of polycarboxylate cements are revealed in Bull. Kanagawa Dent. Coll. (1990), 18(1), 29-32. The polyacids investigated exhibit a polydispersity ranging from 1.9 to 5.9. To summarize, it is explained that the strength of cements prepared with a poly(acrylic acid) with an average molecular weight of greater than 100 000 with simultaneously the smallest possible molecular weight distribution is very good. Finally, it is emphasized that, in developments in this field, both the average molecular weight and the molecular weight distribution should be considered.

Attempts by the Applicant Company to confirm the reproducibility of this publication have revealed that, e.g., the polyacid according to Sample L82 exhibits a molar mass distribution of 7.4 and not of 1.9 as stated.

The influence of the molecular weight of polyacids on the properties of glass ionomer cements is investigated in J. Dent. Res. (1989), 58(2), 89-94. The polydispersity of the polyacids investigated lies, according to the values cited, in the range from 1.58 to 11.50. The values were obtained by gel permeation chromatography using polyethylene oxide equivalents as external standard.

However, it is essential, for the reproducible determination of molecular weights and molecular weight distributions by gel permeation chromatography, to choose a standard which exhibits the same basic chemical structure as the substance to be measured, since this has a considerable influence on the elution times (J. M. G. Lowie, Chemie und Physik der synthetischen Polymere [Chemistry and Physics of Synthetic Polymers], Vieweg Verlag 1997, Chapter 9.14). It is possible, in particular with comparatively polar polymers, such as polyacids, which exhibit strong tendencies to form associations via hydrogen bonds, in particular with relative measurements against standards with different chemical structures, to obtain measurement results which are seriously in error.

That is why it is an object of the present invention to find polyacids which, while retaining the setting and physical properties of the set cements, in particular together with conventional cement powders, make possible reduced solution viscosities of the liquid cement and consequently improved consistencies during the mixing operation.

This object is achieved through the use of polyacids or of polyacids together with salts of polyacids with a molar mass distribution of [sic] Mw/Mn between 1.0 and 1.7, preferably between 1.0 and 1.5, particularly preferably between 1.0 and 1.3, in dental materials or the preparation of dental materials comprising polyacids with such a molar mass distribution.

The term “Mw” is to be understood, within the meaning of this application, as the weight-average molecular weight determined by means of aqueous gel permeation chromatography (GPC), for which applies:

$$Mw = \frac{{\sum }_{i} {n}_{i} {M}_{i}^{2}}{{\sum }_{i} {n}_{i} {M}_{i}}$$

in which nᵢ=number of the polymer chains and Mᵢ=molar mass of the polymer chain.

The term “Mn” is to be understood, within the meaning of this application, as the number-average molecular weight determined by means of aqueous GPC, for which applies:

$$Mn = \frac{{\sum }_{i} {n}_{i} {M}_{i}}{{\sum }_{i} {n}_{i}} = \frac{{\sum }_{i} {n}_{i} {M}_{i}}{n}$$

in which n=total number of the polymer chains in a sample.

The term “dental materials” is to be understood, within the framework of this application, as in particular cements, such as zinc polycarboxylate cements, glass ionomer cements or resin-modified glass ionomer cements, and compomers, provided that they can be formulated with aqueous polyacid solutions. The dental materials are curable materials, i.e. materials which in accordance with the requirements change in a period of time of 30 sec to 30 min, preferably of 2 min to 10 min, from a viscous to a nonviscous condition.

The curing reaction can; for example, be brought about by a cement reaction, a crosslinking reaction and/or a polymerization reaction.

A viscous condition, in contrast to a nonviscous condition, in the above meaning then exists if the material can be processed or applied with application devices familiar to dentists, such as a spatula, syringe or mixing capsule.

The term “polyacids” is to be understood as meaning polymers and copolymers which exhibit more than three acid groups per polymer molecule. The polymers or copolymers can in addition also exhibit other functional groups. Polyacids according to the present invention are not to be understood as individual substances but as mixtures of the most varied individual molecules which in each case exhibit varying molar masses.

The term “acid groups” is to be understood as meaning in particular carboxylic acid groups, phosphonic acid groups, phosphoric acid groups or sulfonic acid groups.

The terms “to include” and “to comprise” within the meaning of the present invention introduce an enumeration of characteristics which is not comprehensive. The expression “one” is equivalent to the statement of “at least one”.

The polyacids used according to the invention exhibit a molar mass Mw ranging from 1 000 to 500 000, preferably ranging from 2 000 to 100 000, particularly preferably ranging from 5 000 to 80 000.

Polyacids exhibiting the molar mass distribution according to the invention have, for example, at a concentration of 38 to 47% in water, a viscosity of 0.49 to 4.23 Pa.s, preferably, at a concentration of 42 to 46% in water, a viscosity of 1.55 to 3.11 Pa.s, the viscosity being determined with a PK100 (Haake) viscometer at 23° C.

The fact that the object described is achieved with the molar mass distribution according to the invention is surprising because, according to previous information, the high molar masses of a polyacid with a broad distribution were regarded as essential for cement strengths (H. J. Posser et al., J. Dent. Res., 1986, and J. Dent. (1977), 5(2), 117-20), while the low molar masses drastically reduce the viscosity of the polyacid solution (H. G. Elias, Makromoleküle [Macromolecules], Volume 1, Hüthing & Wepf Verlag).

The disadvantages relating back to branchings, such as poor accessibility of the acid groups, are avoided in a particularly simple way by the use of unbranched polyacids with a narrow molar mass distribution.

The polyacids which can be used according to the invention preferably exhibit repeat units of the formula (1) occurring either as sole repeat units or with additional repeat units, the repeat units in copolymers being able to be randomly or alternately arranged along the main polymer chain:
—CR¹R²—CR³R⁴— (1)
C=carbon;
R¹═COOH, PO₃H₂, OPO₃H₂, SO₃H₂;
R²═H, CH₃, C₂H₅ or CH₂COOH;
R³═H;
R⁴═H, COOH, COOR⁵, in which R⁵ is taken from the following group: linear, branched or cyclic alkyl residue with 1 to 12 C atoms, preferably CH₃, C₂H₅, C₃H₇, C₄H₉; substituted or unsubstituted aryl residue with 6 to 18 C atoms, preferably phenyl or benzyl; linear, branched or cyclic alkyl residue with 1 to 12 C atoms functionalized with 1 to 5 heteroatoms from the group consisting of N, O and S, preferably CH₃O, C₂H₄OR⁶, in which R⁶ represents an acyl residue, preferably acryl or methacryl.

Preferred representatives of formula (1) are:
1. Polyacrylic acid 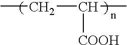
2. Copolymers of polyacrylic acid with more than 30 mol %, preferably more than 50 mol %, of acrylic acid units;
3. Polymers and copolymers with more than 30 mol %, preferably more than 50 mol %, of units from the group: maleic acid, methacrylic acid, fumaric acid, itaconic acid, vinylphosphonic acid, vinylidenediphosphonic acid, vinylsulfonic acid or vinyl phosphate.

Comonomers for this are preferably taken from the group: methacrylic acid, maleic acid, maleic anhydride, fumaric acid, itaconic acid, itaconic anhydride, glutaconic acid, citridic acid, citraconic acid, methaconic [sic] acid, tiglic acid, crotonic acid, muconic acid, isocrotonic acid, 3-butenoic acid, cinnamic acid, styrenecarboxylic acid, vinylphthalic acid, abietinic acid, styrenesulfonic acid, styrene-phosphonic acid, 1-phenylvinylphosphonic acid, vinylphosphonic acid, vinylidenediphosphonic acid, vinylsulfonic acid, vinyl phosphate, other monomers with acid functional groups and substituted derivatives thereof, especially esters or amides or imides of the acids mentioned with up to 10 C atoms in the alkoxide or amine or imine residue, preferably acrylic esters, methacrylic esters, maleic esters or corresponding amides or imides, for example maleimide, for example methyl acrylate, ethyl acrylate, n-propyl acrylate, isopropyl acrylate, n-butyl acrylate, isobutyl acrylate, tert-butyl acrylate, N-methylmaleimide, N-ethylmaleimide, acrylamide, N-methylacrylamide, N,N-dimethylacrylamide, N-ethylacrylamide, 2-hydroxyethyl acrylate, 2-hydroxyethyl methacrylate, 3-hydroxypropyl acrylate or 3-hydroxypropyl methacrylate.

Other comonomers, additionally or exclusively, can also be included, for example ethylene, butadiene or isoprene.

The comonomers can be incorporated randomly or alternately, preferably randomly.

Preference is likewise given to partially saponified polyacrylic esters, in particular poly(acrylic acid-co-methyl acrylate), poly(acrylic acid-co-ethyl acrylate), poly(acrylic acid-co-propyl acrylate), in which the propyl residue can be either linear or branched, poly(acrylic acid-co-butyl acrylate [sic], in which the butyl residue can be linear or branched, poly(acrylic acid-co-phenyl acrylate), poly(acrylic acid-co-benzyl acrylate), in each case with an incorporation ratio of the comonomers of 1:1 000 to 1 000:1.

It is common to the polyacids used according to the state of the art that they are prepared by means of conventional radical polymerization. In the course of this, materials are inevitably formed having a broad molar mass distribution with Mw/Mn very much greater than 1.7, generally approximately 4 to 6.

Polyacids according to the present invention displaying the molar mass distributions according to the invention can preferably be prepared via anionic polymerization, group transfer polymerization or controlled radical polymerization.

Suitable methods are described in more detail below:

A possible synthesis is carried out via the esters of polyacids, the acids with the molar mass distribution according to the invention being obtained by living anionic or group transfer polymerization of the corresponding monomers and subsequent polymer-analogous saponification of the ester groups.

The term “polymer-analogous reactions” is to be understood, within the meaning of this invention, as generally reactions on the polymer while retaining the degree of polymerization.

For example, esters of polyacrylic acid, such as tert-butyl ester, n-butyl ester, benzyl ester or trimethyl-silyl ester, can be polymerized and subsequently the ester groups can be saponified in a polymer-analogous way, as is described in S. P. Rannard et al., Eur. Polym. J., 29, 407-414 (1993), or in Y. Morishima et al., Macromol. Chem., Rapid Commun., 2, 507-510 (1981). Polyacids with the molar mass distribution according to the invention can be obtained as well through the use of controlled radical polymerization. Polymerization methods based on the following systems are suitable:
N-oxyl radical initiator systems, as described in A. Goto and T. Fukuda, Macromolecules, 1999, 32, 618-623, or Keoshkerian et al., Macromolecules, 1998, 31, 7559-7561;
metal complex/halide systems, as described in K. Matyjaszewski et al., Macromol. Rapid Commun., 20, 341-346 (1999).

Additional applicable methods are reverse atom transfer radical polymerization, as described in G. Moineau et al., Macromolecules, 1998, 31, 545-547, and reversible addition-fragmentation chain transfer (RAFT method), as described in Y. K. Chong et al., Macromolecules, 1999, 32, 2071-2074.

The saponification of polyacid esters is possible according to conventional methods (Houben-Weyl, Methoden der organischen Chemie), without the narrow molar mass distribution changing. Methyl, benzyl or tert-butyl esters, for example, are especially easy to cleave and for this reason are preferred.

Polyacids with the molar mass distribution according to the invention, in the simplest case polyacrylic acid, show, with carboxylate cements and glass ionomer cements, identical strengths with only 25 to 30% of the solution viscosity. The number-average molar masses Mn are in this case comparable with those of conventional polyacids with a broad distribution from dental materials, for example polyacrylic acid.

Curable dental materials or cements can be prepared with the polyacids described, which dental materials or cements have a compressive strength ranging from 220 to 250 MPa (measured according to ISO 9917), a bending strength ranging from 35 to 45 MPa (measured analogously to ISO 4049 with test specimens 12 mm long) and/or a surface hardness ranging from 400 to 500 MPa (measured according to DIN 53456).

Powder/liquid ratios which can be used for curable dental materials according to the invention obtainable by mixing a powder with a liquid range from 0.7 to 6.0, preferably from 1.0 to 5.0, particularly preferably from 1.5 to 3.5.

With the number-average molar mass Mn, the polymer chains of varying lengths are weighted in accordance with their number in the averaging (Lehrbücher der Polymerchemie [Manuals of Polymer Chemistry], for example H. G. Elias, Makromoleküle [Macromolecules], Volume 1, Hüthing & Wepf-Verlag).

The cations of the salts of the polyacids which can be used according to the invention are taken from the group: alkali metal elements, alkaline earth metal elements, zinc, aluminum, scandium, yttrium or lanthanum. Na, Ca and Al salts are preferred in this connection.

The polyacids described in the framework of this invention are used in dental materials in order, in comparison with dental materials from the state of the art, in which polyacids with a high value of the molar mass distribution are used, to make possible a decrease in the viscosity while retaining the physical parameters of the cured dental material. In particular, the compressive strength and the bending strength, in addition to the surface hardness, are not reduced, in many cases are even improved, and the viscosity of the mixed dental material is reduced before the beginning of the curing.

The polyacids or salts of polyacids, provided that they are sufficiently soluble, can, according to the field of application, be used as liquid or as solid, for example obtained through freeze drying or spray drying.

Preferred dental materials in the framework of this invention are either formulated with a single component, such as compomers, or with two or more components, such as carboxylate cements, glass ionomer cements and resin-modified glass ionomer cements, in which the liquid components are stored separately from the solid components and are mixed immediately before application.

Particular preference is given to cements which, in the case of glass ionomer cements, for example, can include the following constituents:
(A) 1 to 60% by weight, preferably 5 to 40% by weight, particularly preferably 10 to 30% by weight, of polyacids or polyacids together with salts of polyacids with the molar mass distribution according to the invention;
(B) 35 to 80% by weight, preferably 50 to 70% by weight, of fillers;
(C) 0 to 20% by weight, preferably 1 to 10% by weight, of additives and auxiliaries;
(D) 5 to 40% by weight, preferably 9 to 30% by weight, of water.

The term “fillers” of the component (B) is to be understood as mainly reactive or nonreactive solids.

Suitable examples are reactive fluoroaluminosilicate glasses from DE 20 61 513 A1, DE 20 65 824 A1, or reactive glasses which, on the surface, in comparison with the average composition, are depleted in calcium ions, as described in DE 29 29 121 A1.

The last-named glasses are especially preferred and can exhibit the following composition:

| Constituent | Calculated as | % by weight |
|---|---|---|
| Si | SiO₂ | 20 to 60 |
| Al | Al₂O₃ | 10 to 50 |
| Ca | CaO | 1 to 40 |
| F | F | 1 to 40 |
| Na | Na₂O | 0 to 10 |
| P | P₂O₅ | 0 to 10 |

and a total of 0 to 20% by weight, calculated as oxides, of B, Bi, Zn, Mg, Sn, Ti, Zr, La or other trivalent lanthanides, K, W, Ge, and other additives, which do not impair the properties and are physiologically completely harmless.

In addition to the reactive glasses described above, inert fillers, such as quartz, can be used.

The term “component (C)” is to be understood as, for example, additives for accelerating and improving the curing, as are known from DE 2 319 715 A1. Preferably, chelating agents in the form of low molecular weight acid molecules, such as tartaric acid, are added.

Coloring pigments and other auxiliaries usual in the field of glass ionomer cements, for example for improving the miscibility, are also to be understood under “component (C)”.

Apart from their use in conventional glass ionomer cements, the polyacids with the molar mass distribution according to the invention are also suitable for use in carboxylate cements.

In this connection, the compositions include, for example, the following constituents:
(A) 1 to 60% by weight, preferably 5 to 40% by weight, particularly preferably 10 to 30% by weight, of polyacids or polyacids together with salts of polyacids with the molar mass distribution according to the invention;
(C) 0 to 20% by weight, preferably 1 to 10% by weight, of additives and auxiliaries;
(D) 10 to 40% by weight, preferably 15 to 30% by weight, of water;
(E) 30 to 80% by weight, preferably 44 to 70% by weight, of zinc oxide.

Compomers or resin-modified glass ionomer cements comprising the polyacids with the molar mass distribution according to the invention include, for example, the following components:
(A) 1 to 75% by weight, preferably 2 to 69.9% by weight, particularly preferably 10 to 30% by weight, of polyacids or polyacids together with salts of polyacids with the molar mass distribution according to the invention;
(D) 5 to 40% by weight, preferably 10 to 30% by weight, of water;
(F) 8.9 to 70% by weight, preferably 10 to 60% by weight, of one or more radically polymerizable monomers;
(G) 10 to 90% by weight, preferably 15 to 87.9% by weight, of fillers;
(H) 0.1 to 5% by weight, preferably 0.5 to 3% by weight, of initiators and optionally activators;
(I) 0 to 30% by weight, preferably 0.1 to 20% by weight, of additives, optionally pigments, thixotropic agents, plasticizers.

Mono-, di- or polyfunctional ethylenically unsaturated compounds, preferably based on acrylate and/or methacrylate, are used as component (F). These can comprise both monomeric and polymolecular oligomeric or polymeric acrylates. In addition, they can be used in the formulations alone or as mixtures.

Suitable monomers are, for example, the acrylic and methacrylic esters of mono-, di- or polyfunctional alcohols. The following are mentioned as examples: 2-hydroxyethyl (meth)acrylate, methyl (meth)acrylate, isobutyl (meth)acrylate, ethylene glycol di(meth)acrylate, triethylene glycol di(meth)acrylate (TEGDMA), hexanediol di(meth)acrylate, dodecanediol di(meth)acrylate and trimethylolpropane tri(meth)acrylate.

Others which can advantageously be used are bisphenol A di(meth)acrylate and the ethoxylated or propoxylated di(meth)acrylates derived therefrom. In addition, the monomers described in U.S. Pat. No. 3,066,112 A, based on bisphenol A (meth)acrylate and glycidyl (meth)acrylate or their derivatives arising from addition of isocyanates, are suitable.

The diacrylic and dimethacrylic esters of bis(hydroxy-methyl)tricyclo[5.2.1.0^{2,6}] decane mentioned in DE 28 16 823 C1 and the diacrylic and dimethacrylic esters of the compounds of bis(hydroxy-methyl)tricyclo[5.2.1.0^{2,6}] decane extended with 1 to 3 ethylene oxide and/or propylene oxide units are also especially suitable.

Urethane (meth)acrylates, such as 7,7,9-trimethyl-4,13-dioxo-5,12-diazahexadecane-1,16-dioxy [sic] dimethacrylate (UDMA), can also be a constituent of this component.

Fillers according to component (G) can be inorganic fillers, for example quartz, glass powder, water-insoluble fluorides, such as CaF₂, silica gels and silicic acid, especially pyrogenic silica gel, and their granulates. Cristobalite, calcium silicate, zirconium silicate, zeolites, including molecular sieves, metal oxide powders, such as aluminum or zinc oxides or their mixed oxides, barium sulfate, yttrium fluoride or calcium carbonate can also be used as fillers.

Fluoride-releasing fillers, for example complex inorganic fluorides of the general formula AₙMFₘ, as described in DE 44 45 266 A1, can also be used or added. A represents a mono- or polyvalent cation, M represents a metal from the main group or subgroup III, IV or V, n represents an integer from 1 to 3 and m represents an integer from 4 to 6.

Organic fillers can also be a constituent of this component.

Those mentioned by way of example are conventional pearl-shaped polymers and copolymers based on methyl methacrylate, which, for example, are available from Röhm under the name “Plexidon” or “Plex”.

For improved incorporation in the polymer matrix, it can be advantageous to render hydrophobic, using a silane, the fillers mentioned and optionally additives opaque to X-rays. The amount of the silane used usually amounts to 0.5 to 10% by weight, with reference to inorganic fillers, preferably 1 to 6% by weight, very particularly preferably 2 to 5% by weight, with reference to inorganic fillers. Normal hydrophobing agents are silanes, for example trimethoxymethacryl-oxypropylsilane [sic].

The maximum mean particle size of the preferably inorganic fillers usually amounts to 15 μm, in particular 8 μm. Fillers with a mean particle size of <3 μm are very particularly preferably used.

The term “initiators” according to component (H) is to be understood as initiator systems which bring about the radical polymerization of monomers, for example photoinitiators and/or what are known as redox initiator systems and/or thermal initiators.

Examples of suitable photoinitiators are α-diketones, such as camphorquinone, in conjunction with secondary and tertiary amines, or mono- and bisacylphosphine oxides, such as 2,4,6-trimethylbenzoyldiphenylphosphine oxide and bis (2,6-dichlorobenzoyl) (4-n-propylphenyl)-phosphine oxide. However, other compounds of this type, as are described in EP 0 073 413 A1, EP 0 007 508 A1, EP 0 047 902 A1, EP 0 057 474 A1 and EP 0 184 095 A1, are also suitable.

Organic peroxide compounds together with “activators” are suitable as redox initiator systems. Compounds such as lauroyl peroxide, benzoyl peroxide, p-chlorobenzoyl peroxide and p-methylbenzoyl peroxide are suitable in particular as organic peroxide compounds.

Tertiary aromatic amines, such as the N,N-bis(hydroxyalkyl)-3,5-xylidines known from U.S. Pat. No. 3,541,068, and the N,N-bis(hydroxyalkyl)-3,5-di(tert-butyl)anilines known from DE 26 58 530 A1, especially N,N-bis(β-hydroxybutyl)-3,5-di(tert-butyl)aniline, and N,N-bis(hydroxyalkyl)-3,4,5-trimethylanilines, for example, are suitable as activators.

Highly suitable activators are also the barbituric acids and barbituric acid derivatives described in DE 14 95 520 A1 and the malonyl sulfamides described in EP 0 059 451 A1. Preferred malonyl sulfamides are 2,6-dimethyl-4-isobutylmalonyl sulfamide, 2,6-diisobutyl-4-propylmalonyl sulfamide, 2,6-dibutyl-4-propylmalonyl sulfamide, 2,6-dimethyl-4-ethylmalonyl sulfamide and 2,6-dioctyl-4-isobutylmalonyl sulfamide.

For further acceleration, the polymerization is in this connection preferably carried out in the presence of heavy metal compounds and ionogenic halogen or pseudo-halogen. Copper is especially suitable as heavy metal and the chloride ion is especially suitable as halide. The heavy metal is suitably used in the form of soluble organic compounds. Likewise, the halide and pseudohalide ions are suitably used in the form of soluble salts, examples which may be mentioned being the soluble amine hydrochlorides and quaternary ammonium chloride compounds.

If the dental materials according to the invention comprise a redox initiator system formed from organic peroxide and activator, peroxide and activator are then preferably present in parts of the dental material according to the invention which are spatially separate from one another, and they are homogeneously mixed with one another only immediately before use. If organic peroxide, copper compound, halide and malonyl sulfamide and/or barbituric acid are present side by side, then it is particularly sensible for the organic peroxide, malonyl sulfamide and/or barbituric acid and the copper compound/halide combination to be present in three constituents spatially separate from one another. For example, the copper compound/halide combination, polymerizable monomers and fillers can be kneaded to a paste and the other components can be kneaded to two separate pastes in the above-described way in each case with a small amount of fillers or in particular thixotropic agents, such as silanized silicic acid, and a plasticizer, for example phthalate. On the other hand, the polymerizable monomers can also be present together with organic peroxide and fillers. Alternatively, organic peroxide, copper compound, halide and malonyl sulfamide and/or barbituric acid can also be split up according to DE 199 28 238 A1.

Soluble organic polymers can be used as representatives of component (I), for example for increasing the flexibility of the materials. Poly(vinyl acetate) and the copolymers based on vinyl chloride/vinyl acetate, vinyl chloride/vinyl isobutyl ether and vinyl acetate/maleic acid dibutyl ether [sic], for example, are suitable. Dibutyl, dioctyl and dinonyl phthalates or adipates and polymolecular polyphthalic and adipic [sic] esters, for example, are highly suitable as additional plasticizers. Modified layered silicates (bentonites) or organic modifying agents, for example based on hydrogenated castor oils, can also be used, in addition to pyrogenic silicic acids, as thixotropic agents. Furthermore, inhibitors, as are described in EP 0 374 824 A1 as component (d), can be included in the formulations as additives.

Furthermore, containers which include dental materials comprising polyacids or polyacids together with salts of polyacids with a molar mass distribution according to the invention, for example capsules, blister packs, application syringes or cannulas, are a subject matter of this invention.

The invention is subsequently illustrated by means of examples, without it being limited in any way thereby. The determination of the molar mass distribution was carried out in this connection via aqueous gel permeation chromatography (GPC) at pH=7 against polyacrylic acid sodium salt standard with the RID6a refractive index detector (Shimadzu). Polyacrylic acid sodium salt standards (PSS) were used for calibration. The measured values were converted to free polyacrylic acid using the factor 0.766. The measurement was carried out at 23° C.

The samples were measured here as 0.05% aqueous solutions in the solvent, a 0.9% by weight aqueous sodium nitrate solution, to which 200 ppm of sodium azide are also added. The solutions were adjusted to pH=7 by addition of sodium hydroxide.

A column combination of Hema3000, HemaBio1000 and HemaBio40 (PSS) was used to measure maximum molecular weights of up to 670 000 g/mol. A column combination of Suprema1000, Hema3000 and HemaBio1000 (PSS) was used with maximum molecular weights of up to 1 100 000 g/mol.

### SYNTHETIC EXAMPLE 1

### Preparation of a Polyacrylic Acid (a)

A mixture of 0.65 g of methyl 2-bromopropionate, 0.28 g of copper(I) bromide, 0.02 g of copper(II) bromide, 0.35 g of N,N,N′,N″,N″-pentamethyldiethylenetriamine (PMDETA) and 100 g of tert-butyl acrylate was dissolved in 100 ml of dimethylformamide in a reaction flask under an argon atmosphere and was heated to 70° C. The polymerization reaction was monitored using ¹H NMR spectroscopy. After 24 hours, the preparation was virtually completely polymerized.

After dissolving the mixture in 500 ml of toluene, washing was carried out with dilute hydrochloric acid and with water, and the organic phase was subsequently concentrated.

The polymer obtained was, in order to cleave the ester groups, dissolved in 500 ml of dioxane and heated for five hours at 100° C. with an excess of concentrated hydrochloric acid. Dioxane and hydrochloric acid were removed by repeated extraction with diethyl ether, concentration on a rotary evaporator, finally with addition of water. The polyacrylic acid was obtained as a colorless aqueous solution which was adjusted to the concentrations given in table 1 (according to titration with lithium hydroxide in the presence of 1% lithium chloride).

### SYNTHETIC EXAMPLE 2

### Preparation of a Polyacrylic Acid (b)

A mixture of 1.22 g of methyl 2-bromopropionate, 0.102 g of copper(I) bromide, 0.250 g of PMDETA and 87 g of methyl acrylate was heated to 80° C. in a reaction flask under an argon atmosphere. After 15 hours, the preparation was virtually completely polymerized. The preparation was dissolved in 500 ml of toluene and extracted with dilute hydrochloric acid and with water. The organic phase was concentrated.

The product was dissolved in 500 ml of dioxane and treated with 100 ml of 4N potassium hydroxide solution. Ester saponification was complete after heating at 80° C. for five hours. The lumpy mixture was diluted with water and treated with an acidic ion exchanger. Repeated extraction with diethyl ether and concentration under vacuum with addition of water gave a yellow-colored aqueous solution of the polyacrylic acid.

The determination of the molar mass distribution was carried out via aqueous GPC at pH=7 against polyacrylic acid sodium salt standard.

### SYNTHETIC EXAMPLE 3

### Preparation of Poly(Acrylic Acid-co-butyl Acrylate) (c)

89.8 g of n-butyl acrylate, 0.288 g of 2,2,6,6-tetramethyl-1-(α-phenethoxy)piperidine (TEMPEP), 0.64 g of α-D-(+)-glucose and 0.64 g of sodium hydrogen-carbonate were mixed together in a reaction flask under an argon atmosphere and were heated at 145° C. for five hours, during which a polymerization conversion of 40% was achieved. The preparation was concentrated under vacuum and extracted several times with methanol. The polymer was obtained as a colorless oil.

For partial saponification, 16.4 g of the poly(butyl acrylate) were dissolved in 160 ml of ethanol and were treated with 10 ml of water and 10.4 g of sodium hydroxide. The preparation was stirred at 50° C. for 24 hours, during which several portions of water were added in order to prevent precipitation of the polymer. The reaction conversion was 96%.

The reaction solution was diluted with water and treated with an acidic ion exchanger. Extraction was subsequently carried out with diethyl ether and the aqueous phase was concentrated on a rotary evaporator. A slightly yellowish, aqueous solution of the poly(acrylic acid-co-butyl acrylate) with an incorporation ratio of 96:4 in favor of acrylic acid (¹H NMR) was obtained.

The determination of the molar mass distribution was carried out via aqueous GPC at pH=7.

### SYNTHETIC EXAMPLE 4

### Preparation of the Sodium Salt of Polyacrylic Acid (a) [sic]

80 g of a 40% aqueous solution of the polyacrylic acid according to synthetic example 1 are treated with a solution of 17.8 g (1 equivalent) of sodium hydroxide in 50 ml of water with stirring and cooling. The preparation is stirred at ambient temperature for a further 3 hours and is subsequently concentrated to dryness on a rotary evaporator. The salt of the polymer is ground up in a mortar and is dried at 110° C. in a drying oven to constant weight.

### SYNTHETIC EXAMPLE 5

### Preparation of the Sodium Salt of Polyacrylic Acid (e)

The synthesis was carried out analogously to synthetic example 2 [sic].

The determination of the molar mass distribution was carried out via aqueous GPC at pH=7 against polyacrylic acid sodium salt standard.

### USE EXAMPLES

The polyacids according to the preparation examples were tested in the application for dental carboxylate cements and glass ionomer cements.

The procedure used to determine the compressive strengths of the set cements was that of the ISO 9917 standard. Bending strengths were determined using the ISO 4049 standard, in which, however, test specimens with a length of 12 mm were used. Both the measurement of compressive strengths and that of bending strengths were in each case carried out on a series of 5 test specimens, in which the relative error was approximately ±10% per measured value. Surface hardnesses were determined according to DIN 53456.

The viscosities of the aqueous polyacid solutions were measured with a PK 100 viscometer from Haake at 23° C.

Concentrations were determined by titration with lithium hydroxide in the presence of 1% by weight of lithium chloride.

The stated polyacids were spatulated either with the powder part of the carboxylate cement Durelon® (ESPE Dental AG, Seefeld, Germany), including more than 90% by weight of zinc oxide, or a standard glass ionomer powder based on a calcium strontium fluoroaluminosilicate glass (glass ionomer cement glass) (Diamond Carve Powder, Kemdent, England). In each case, the liquid of the product Durelon® already mentioned above, a polyacid with a molar mass distribution Mw/Mn=5.0, was used as comparative example. The results are summarized in table 2.

TABLE 1
Properties of the polyacids with a molar mass
distribution according to the invention and
of the comparative example

| | Concentration | Viscosity | | |
|---|---|---|---|---|
| Polyacid | [%] | [Pa · s] | Mn | Mw/Mn |
| Comparison | 42 | 8.13 | 12 000 | 5.0 |
| (a) | 40 | 0.57 | 13 000 | 1.1 |
| | 42 | 1.55 | | |
| | 45 | 2.68 | | |
| | 47 | 3.79 | | |
| (b) | 38 | 0.49 | 12 500 | 1.5 |
| | 42 | 1.72 | | |
| | 44 | 2.58 | | |
| | 47 | 4.23 | | |
| (c) | 46 | 3.11 | 16 000 | 1.3 |
| (d) | 41 | 1.98 | 15 100 | 1.4 |
| (e) | 38 | 2.73 | 60 000 | 1.2 |

The viscosity of the polyacids exhibiting the molar mass distributions according to the invention is, at comparable concentration and comparable number-average molecular weight Mn, lower than that of the comparative example.

TABLE 2
Strength values of the use examples

| | Concen- | | Powder/ | Compressive | Bending |
|---|---|---|---|---|---|
| | tration | Powder | liquid | strength | strength |
| Polyacid | [%] | component | ratio | [MPa] | [MPa] |
| (a) | 42 | Durelon | 1.5 | 70 | |
| (a) | 42 | Durelon | 2.5 | 97 | |
| Comparison | 42 | Durelon | 1.5 | 75 | |
| Comparison | 42 | Durelon | 2.5 | 95 | |
| (a) | 42 | GIC glass | 3.5 | 240 | 43 |
| Comparison | 42 | GIC glass | 3.5 | 237 | 42 |
| (b) | 42 | Durelon | 1.5 | 72 | |
| (b) | 42 | Durelon | 2.5 | 96 | |
| (b) | 42 | GIC glass | 3.5 | 243 | 41 |
| Comparison | 42 | Durelon | 1.5 | 75 | |
| Comparison | 42 | Durelon | 2.5 | 95 | |
| Comparison | 42 | GIC glass | 3.5 | 237 | 42 |
| (e) | 38 | GIC glass | 3.5 | 232 | 42 |
| (c) | 46 | Durelon | 1.5 | 74 | |
| (c) | 46 | GIC glass | 3.5 | 246 | 44 |

| | | | | |
|---|---|---|---|---|
| Comparison | 46 | Durelon | 1.5 | (*) |
| Comparison | 46 | GIC glass | 3.5 | |

GIC = glass ionomer cement
(*) The viscosity of the comparative polyacid is too high for suitable test specimens to be prepared.

The strength values of the dental materials including polyacids with a molar mass distribution according to the invention lie in the same range as the values of the comparative materials.

The surface hardnesses of the dental materials according to the invention, when glass ionomer cement glass is used, lie in the range from 400 to 500 MPa and consequently at the same level as when the comparative polyacid is used.

To summarize, polyacids with a molar mass distribution according to the invention make possible the formulation of dental materials of reduced viscosity with, in comparison with materials of the state of the art, unchanged strength values.

1-10. (canceled)

11. A curable dental material, comprising:
at least one polyacid, or at least one polyacid with at least one salt of at least one polyacid
wherein the at least one polyacid or at least one polyacid with at least one salt of at least one polyacid has a molar mass distribution Mw/Mn between 1.0 and 1.7,
wherein the at least one polyacid or at least one polyacid with at least one salt of at least one polyacid has a molecular weight Mw ranging from 1,000 to 500,000,
wherein the at least one polyacid or at least one polyacid with at least one polyacid salt is optionally prepared via at least one of anionic polymerization, group transfer polymerization, and controlled radical polymerization.

12. The curable dental material as claimed in claim 11, wherein said at least one polyacid or at least one polyacid with at least one salt of at least one polyacid is linear, unbranched, and noncrosslinked.

13. The curable dental material as claimed in claim 11, wherein said a molar mass distribution Mw/Mn is between 1.0 and 1.5.

14. The curable dental material as claimed in claim 11, wherein said a molar mass distribution Mw/Mn is between 1.0 and 1.3.

15. The curable dental material as claimed in claim 11, wherein the polyacid or polyacid salt comprises repeat units of formula (1),
wherein the repeat units occur either as sole repeat units, or with optional additional repeat units to form a copolymer, and the repeat units in the copolymer are randomly or alternately arranged along a main polymer chain:
—CR^{I}R²—CR³R⁴— (1)
wherein C=carbon;
R¹═COOH, PO₃H₂, OPO₃H₂, or SO₃H₂;
R²═H, CH₃, C₂H₅, or CH₂COOH;
R³═H; and
R⁴═H, COOH, or COOR⁵ in which R⁵ is selected from the group consisting of:
linear, branched, or cyclic alkyl residues with 1 to 12 C atoms;
substituted or unsubstituted aryl residues with 6 to 18 C atoms;
linear, branched or cyclic alkyl residues with 1 to 12 C atoms,
wherein at least one C atom is functionalized with 1 to 5 heteroatoms selected from the group consisting of N, O, and S.

16. The curable dental material as claimed in claim 15, wherein R⁴ is at least one of CH₃O and C₂H₄OR⁶, wherein R⁶ is an acyl residue.

17. The curable dental material as claimed in claim 16, wherein R⁶ is acryl or methacryl.

18. The curable dental material as claimed in claim 11, wherein the polyacid is at least one of:
polyacrylic acid;
poly(acrylic acid-alt-maleic acid);
poly(acrylic acid-co-maleic acid) with an incorporation ratio of the comonomers of 1:1,000 to 1,000:1;
poly(acrylic acid-co-itaconic acid) with an incorporation ratio of the comonomers of 1:1,000 to 1,000:1;
poly(acrylic acid-co-fumaric acid) with an incorporation ratio of the comonomers of 1:1,000 to 1,000:1; and
partially saponified polyacrylic esters.

19. A curable dental material, comprising:
(A) 1 to 60% by weight of at least one polyacid or at least one polyacid and salt of at least one polyacid, wherein the at least one polyacid or at least one polyacid and salt of at least one polyacid have a molar mass distribution Mw/Mn between 1.0 and 1.7;
(B) 35 to 80% by weight of at least one filler;
(C) 0 to 20% by weight of at least one of an additive and an auxiliary; and
(D) 5 to 40% by weight of water,
wherein the polyacid is optionally prepared via at least one of anionic polymerization, group transfer polymerization, and controlled radical polymerization.

20. A curable dental material, comprising:
(A) 1 to 60% by weight of polyacids or polyacids and salts of polyacids with a molar mass distribution Mw/Mn between 1.0 and 1.7;
(C) 0 to 20% by weight of at least one of an additive and an auxiliary;
(D) 10 to 40% by weight of water; and
(E) 30 to 80% by weight of zinc oxide;
wherein the polyacid is optionally prepared via at least one of anionic polymerization, group transfer polymerization, and controlled radical polymerization.

21. The curable dental material as claimed in claim 20 wherein said a molar mass distribution Mw/Mn is between 1.0 and 1.5.

22. The curable dental material as claimed in claim 20, wherein said a molar mass distribution Mw/Mn is between 1.0 and 1.3.

23. A curable dental material, comprising:
(A) 1 to 75% by weight of at least one polyacid or at least one polyacid and salt of at least one polyacid with a molar mass distribution Mw/Mn between 1.0 and 1.7;
(D) 5 to 40% by weight of water;
(F) 8.9 to 70% by weight of at least one radically polymerizable monomer;
(G) 10 to 90% by weight of at least one filler;
(H) 0.1 to 5% by weight of at least one initiator and optionally at least one activator; and
(I) 0 to 30% by weight of at least one additive;
wherein the polyacid is optionally prepared via at least one of anionic polymerization, group transfer polymerization, and controlled radical polymerization.

24. The curable dental material as claimed in claim 23, wherein the additive comprises at least one of a pigment, a thixotropic agent, and a plasticizer.

25. A method of treating a patient in need of dental material, comprising providing to a patient in need thereof a sufficient amount of a curable dental material, wherein the curable dental material comprises at least one polyacid or at least one polyacid and at least one salt of at least one polyacid
wherein the at least one polyacid or at least one polyacid with at least one salt of at least one polyacid has a molar mass distribution Mw/Mn between 1.0 and 1.7,
wherein the polyacid is optionally prepared via at least one of anionic polymerization, group transfer polymerization, and controlled radical polymerization; and
curing said dental material.

26. A kit, comprising at least one curable dental material, wherein the dental material comprises
at least one polyacid or
at least one polyacid and at least one salt of at least one polyacid
wherein said at least one polyacid or at least one polyacid with at least one salt of at least one polyacid has a molar mass distribution Mw/Mn between 1.0 and 1.7,
wherein the polyacid or polyacid with at least one polyacid salt is optionally prepared via at least one of anionic polymerization, group transfer polymerization, and controlled radical polymerization, and instructions for using the curable dental material.

27. The kit as claimed in claim 26, wherein the kit further comprises at least one of a container and a capsule.

28. A composition of matter, comprising:
at least one polyacid, or at least one polyacid with at least one salt of at least one polyacid,
wherein said at least one polyacid or at least one polyacid with at least one salt of at least one polyacid has a molar mass distribution Mw/Mn between 1.0 and 1.7,
wherein the polyacid has a molecular weight Mw ranging from 1,000 to 500,000.

29. The composition of matter as claimed in claim 28, wherein the at least one polyacid, or at least one polyacid with at least one salt of at least one polyacid, is linear, unbranched, and noncrosslinked.

30. A method of preparing a composition of matter, comprising:
preparing at least one polyacid, or at least one polyacid with at least one polyacid salt via at least one of anionic polymerization, group transfer polymerization, and controlled radical polymerization;
obtaining at least one polyacid or at least one polyacid with at least one salt of at least one polyacid with a molar mass distribution Mw/Mn between 1.0 and 1.7, and a molecular weight Mw ranging from 1,000 to 500,000.

31. A method of preparing a curable dental material, comprising:
preparing at least one polyacid, or at least one polyacid with at least one polyacid salt via at least one of anionic polymerization, group transfer polymerization, and controlled radical polymerization;
obtaining at least one polyacid or at least one polyacid with at least one salt of at least one polyacid with a molar mass distribution Mw/Mn between 1.0 and 1.7, and a molecular weight Mw ranging from 1,000 to 500,000;
combining the at least one polyacid, or at least one polyacid with at least one polyacid salt, with water, a filler, optionally an additive, and optionally an auxilliary.
